SEQUENCE LISTING (13 sequences)

SEQ ID NO: 1 - DNA, 2934 bp, synthetic construct
  source 1..2934: mol_type = other DNA
atggagttggggcctcctctggtcctgctgctggccacagtttggcatggtcagggggcccctgtcatcgagcctagtggcccagaactggttgtagagccgggtgaaacggtgaccctgcgatgtgtgagcaatggcagtgtggaatgggatggccccatctctccctactggaccttggaccctgaatctcccggaagcaccctgaccacaagaaacgcgaccttcaaaaacactgggacctaccgttgtaccgagcttgaagaccccatggcaggcagtaccaccatccacttgtatgtcaaagatccggcccactcttggaatttgctggcacaggaggtgacagtggttgagggccaggaagctgtgctgccctgtctgatcactgaccctgcactgaaggacagtgtctcactgatgcgtgaggggggcaggcaggtcttacgcaaaacggtctacttcttctcgccatggcgagggttcattatccgcaaggctaaagtccttgacagcaatacctacgtgtgcaagaccatggtgaatggtagggaatccacctccactggcatctggcttaaggtgaatcgagtccacccagagcccccacagataaaattggagcctagcaagctggtgcggattcgaggggaggctgcgcagatcgtgtgctcggccactaacgccgaagtgggattcaacgttatcctcaaacgtggagacaccaagctggaaatccccctaaacagtgacttccaagataactattataaaaaagtccgggctctcagtctcaacgctgtggacttccaagacgctggcatatattcttgtgtggccagcaatgatgttggcacacgcacggccaccatgaacttccaggtggtggagagtgcctacttaaacttgacctctgagcagagcctcttgcaggaggtgtctgtgggtgacagcctcatcctcacggtccatgcagatgcctaccctagcatacagcattacaactggacctacctaggtccattctttgaagaccagcgcaagcttgagtttatcacccaaagggccatatacaggtacacattcaagctctttctgaaccgtgtaaaggcctcagaggcgggccagtacttcttaatggcacaaaacaaggcaggctggaataatctgacctttgagctcaccctgcgatatcccccagaggtcagtgttacatggatgcctgtgaatggctctgatgtcctgttctgtgacgtctctgggtaccctcagcccagcgtgacatggatggagtgcaggggccacaccgataggtgtgatgaagcccaggctttgcaggtttggaatgacacccaccctgaagtcctgagtcagaagcccttcgacaaagtgatcattcagagccagctgcccattgggaccttaaaacacaacatgacttatttttgcaaaacccacaacagtgtgggtaacagctctcagtacttcagggccgtctccctaggacaaagcaagcagctccccgatgagtccctcttcactccggtggtggtggcctgtatgtctgtcatgtctctgctggtgctactgctgttgctgctcttgtacaagtacaagcagaagccgaagtaccaggtgcgctggaagatcatcgagagatacgaaggcaatagctacaccttcattgaccctactcagttgccctacaatgagaagtgggagttccctcggaacaacctgcagtttggtaagactctaggagccggtgcctttgggaaggtggtggaggctacagcctttggtctgggcaaagaagatgcagtgctgaaggtggctgtgaagatgctaaagtccacggctcatgctgatgagaaggaggccctgatgtcagagctgaagatcatgagtcacctgggacagcacgagaatatagtcaacctcttgggagcctgtactcacggaggacctgtcctggtcatcactgaatactgctgctatggagacctactcaactttctccgaaggaaggccgaggctatgctaggacccagcctgagtcctggtcaggactccgagggagactccagctacaagaacatccacctggagaagaaatatgtgcgcagggacagtggcttctccagtcagggtgtagacacctacgtggagatgaggcctgtctcgacttcttcaagtgactccttctttaagcaagatctggacaaagaggccagccggcccctggagctctgggacctgctccacttctccagccaagtggctcagggcatggccttccttgcttctaaaaactgcatccaccgggacgtagcagctcgaaacgtgctgttgaccagcggacatgtggccaagattggggactttggactggctagggacatcatgaatgactccaactatgttgtcaagggcaatgcccgcctgcctgtaaagtggatggccccagagagcatctttgactgcgtctacacagttcagagtgatgtgtggtcctacggcatcctcctctgggagatcttctcgcttggtctgaacacctaccccggcatcctagtgaacaacaagttctacaaactggtgaaggatggataccaaatggcccagcctgtatttgcaccgaagaacatatacagcatcatgcagtcctgctgggacctggagcctaccagaagacccaccttccaacagatctgcttcctcctccaggagcaggcccgactggagaggagagaccaggactatgctaacctgccaagcagcggtggcagcagcggcagtgacagtggtggtggcagcagcggtggcagcagcagtgagccagaagaggagagctccagtgaacacctggcctgctgtgagccaggggacatcgcccagcccctgctgcagcctaacaactaccagttctgctga

SEQ ID NO: 2 - protein, 977 aa, synthetic construct
MELGPPLVLLLATVWHGQGAPVIEPSGPELVVEPGETVTLRCVSNGSVEWDGPISPYWTLDPESPGSTLTTRNATFKNTGTYRCTELEDPMAGSTTIHLYVKDPAHSWNLLAQEVTVVEGQEAVLPCLITDPALKDSVSLMREGGRQVLRKTVYFFSPWRGFIIRKAKVLDSNTYVCKTMVNGRESTSTGIWLKVNRVHPEPPQIKLEPSKLVRIRGEAAQIVCSATNAEVGFNVILKRGDTKLEIPLNSDFQDNYYKKVRALSLNAVDFQDAGIYSCVASNDVGTRTATMNFQVVESAYLNLTSEQSLLQEVSVGDSLILTVHADAYPSIQHYNWTYLGPFFEDQRKLEFITQRAIYRYTFKLFLNRVKASEAGQYFLMAQNKAGWNNLTFELTLRYPPEVSVTWMPVNGSDVLFCDVSGYPQPSVTWMECRGHTDRCDEAQALQVWNDTHPEVLSQKPFDKVIIQSQLPIGTLKHNMTYFCKTHNSVGNSSQYFRAVSLGQSKQLPDESLFTPVVVACMSVMSLLVLLLLLLLYKYKQKPKYQVRWKIIERYEGNSYTFIDPTQLPYNEKWEFPRNNLQFGKTLGAGAFGKVVEATAFGLGKEDAVLKVAVKMLKSTAHADEKEALMSELKIMSHLGQHENIVNLLGACTHGGPVLVITEYCCYGDLLNFLRRKAEAMLGPSLSPGQDSEGDSSYKNIHLEKKYVRRDSGFSSQGVDTYVEMRPVSTSSSDSFFKQDLDKEASRPLELWDLLHFSSQVAQGMAFLASKNCIHRDVAARNVLLTSGHVAKIGDFGLARDIMNDSNYVVKGNARLPVKWMAPESIFDCVYTVQSDVWSYGILLWEIFSLGLNTYPGILVNNKFYKLVKDGYQMAQPVFAPKNIYSIMQSCWDLEPTRRPTFQQICFLLQEQARLERRDQDYANLPSSGGSSGSDSGGGSSGGSSSEPEEESSSEHLACCEPGDIAQPLLQPNNYQFC

SEQ ID NO: 3 - DNA, 2934 bp, synthetic construct
  source 1..2934: mol_type = other DNA
atggagttggggcctcctctggtcctgctgctggccacagtttggcatggtcagggggcccctgtcatcgagcctagtggcccagaactggttgtagagccgggtgaaacggtgaccctgcgatgtgtgagcaatggcagtgtggaatgggatggccccatctctccctactggaccttggaccctgaatctcccggaagcaccctgaccacaagaaacgcgaccttcaaaaacactgggacctaccgttgtaccgagcttgaagaccccatggcaggcagtaccaccatccacttgtatgtcaaagatccggcccactcttggaatttgctggcacaggaggtgacagtggttgagggccaggaagctgtgctgccctgtctgatcactgaccctgcactgaaggacagtgtctcactgatgcgtgaggggggcaggcaggtcttacgcaaaacggtctacttcttctcgccatggcgagggttcattatccgcaaggctaaagtccttgacagcaatacctacgtgtgcaagaccatggtgaatggtagggaatccacctccactggcatctggcttaaggtgaatcgagtccacccagagcccccacagataaaattggagcctagcaagctggtgcggattcgaggggaggctgcgcagatcgtgtgctcggccactaacgccgaagtgggattcaacgttatcctcaaacgtggagacaccaagctggaaatccccctaaacagtgacttccaagataactattataaaaaagtccgggctctcagtctcaacgctgtggacttccaagacgctggcatatattcttgtgtggccagcaatgatgttggcacacgcacggccaccatgaacttccaggtggtggagagtgcctacttaaacttgacctctgagcagagcctcttgcaggaggtgtctgtgggtgacagcctcatcctcacggtccatgcagatgcctaccctagcatacagcattacaactggacctacctaggtccattctttgaagaccagcgcaagcttgagtttatcacccaaagggccatatacaggtacacattcaagctctttctgaaccgtgtaaaggcctcagaggcgggccagtacttcttaatggcacaaaacaaggcaggctggaataatctgacctttgagctcaccctgcgatatcccccagaggtcagtgttacatggatgcctgtgaatggctctgatgtcctgttctgtgacgtctctgggtaccctcagcccagcgtgacatggatggagtgcaggggccacaccgataggtgtgatgaagcccaggctttgcaggtttggaatgacacccaccctgaagtcctgagtcagaagcccttcgacaaagtgatcattcagagccagctgcccattgggaccttaaaacacaacatgacttatttttgcaaaacccacaacagtgtgggtaacagctctcagtacttcagggccgtctccctaggacaaagcaagcagctccccgatgagtccctcttcactccggtggtggtggcctgtatgtctgtcatgtctctgctggtgctactgctgttgctgctcttgtacaagtacaagcagaagccgaagtaccaggtgcgctggaagatcatcgagagatacgaaggcaatagctacaccttcattgaccctactcagttgccctacaatgagaagtgggagttccctcggaacaacctgcagtttggtaagactctaggagccggtgcctttgggaaggtggtggaggctacagcctttggtctgggcaaagaagatgcagtgctgaaggtggctgtgaagatgctaaagtccacggctcatgctgatgagaaggaggccctgatgtcagagctgaagatcatgagtcacctgggacagcacgagaatatagtcaacctcttgggagcctgtactcacggaggacctgtcctggtcatcactgaatactgctgctatggagacctactcaactttctccgaaggaaggccgaggctatgctaggacccagcctgagtcctggtcaggactccgagggagactccagctacaagaacatccacctggagaagaaatatgtgcgcagggacagtggcttctccagtcagggtgtagacacctacgtggagatgaggcctgtctcgacttcttcaagtgactccttctttaagcaagatctggacaaagaggccagccggcccctggagctctgggacctgctccacttctccagccaagtggctcagggcatggccttccttgcttctaaaaactgcatccaccgggacgtagcagctcgaaacgtgctgttgaccagcggacatgtggccaagattggggactttggactggctagggacatcatgaatgactccaactatgttgtcaagggcaatgcccgcctgcctgtaaagtggatggccccagagagcatctttgactgcgtctacacagttcagagtgatgtgtggtcctacggcatcctcctctgggagatcttctcgcttggtctgaacccctaccccggcatcctagtgaacaacaagttctacaaactggtgaaggatggataccaaatggcccagcctgtatttgcaccgaagaacatatacagcatcatgcagtcctgctgggacctggagcctaccagaagacccaccttccaacagatctgcttcctcctccaggagcaggcccgactggagaggagagaccaggactatgctaacctgccaagcagcggtggcagcagcggcagtgacagtggtggtggcagcagcggtggcagcagcagtgagccagaagaggagagctccagtgaacacctggcctgctgtgagccaggggacatcgcccagcccctgctgcagcctaacaactaccagttctgctga

SEQ ID NO: 4 - protein, 977 aa, synthetic construct
MELGPPLVLLLATVWHGQGAPVIEPSGPELVVEPGETVTLRCVSNGSVEWDGPISPYWTLDPESPGSTLTTRNATFKNTGTYRCTELEDPMAGSTTIHLYVKDPAHSWNLLAQEVTVVEGQEAVLPCLITDPALKDSVSLMREGGRQVLRKTVYFFSPWRGFIIRKAKVLDSNTYVCKTMVNGRESTSTGIWLKVNRVHPEPPQIKLEPSKLVRIRGEAAQIVCSATNAEVGFNVILKRGDTKLEIPLNSDFQDNYYKKVRALSLNAVDFQDAGIYSCVASNDVGTRTATMNFQVVESAYLNLTSEQSLLQEVSVGDSLILTVHADAYPSIQHYNWTYLGPFFEDQRKLEFITQRAIYRYTFKLFLNRVKASEAGQYFLMAQNKAGWNNLTFELTLRYPPEVSVTWMPVNGSDVLFCDVSGYPQPSVTWMECRGHTDRCDEAQALQVWNDTHPEVLSQKPFDKVIIQSQLPIGTLKHNMTYFCKTHNSVGNSSQYFRAVSLGQSKQLPDESLFTPVVVACMSVMSLLVLLLLLLLYKYKQKPKYQVRWKIIERYEGNSYTFIDPTQLPYNEKWEFPRNNLQFGKTLGAGAFGKVVEATAFGLGKEDAVLKVAVKMLKSTAHADEKEALMSELKIMSHLGQHENIVNLLGACTHGGPVLVITEYCCYGDLLNFLRRKAEAMLGPSLSPGQDSEGDSSYKNIHLEKKYVRRDSGFSSQGVDTYVEMRPVSTSSSDSFFKQDLDKEASRPLELWDLLHFSSQVAQGMAFLASKNCIHRDVAARNVLLTSGHVAKIGDFGLARDIMNDSNYVVKGNARLPVKWMAPESIFDCVYTVQSDVWSYGILLWEIFSLGLNPYPGILVNNKFYKLVKDGYQMAQPVFAPKNIYSIMQSCWDLEPTRRPTFQQICFLLQEQARLERRDQDYANLPSSGGSSGSDSGGGSSGGSSSEPEEESSSEHLACCEPGDIAQPLLQPNNYQFC

SEQ ID NO: 5 - DNA, 17560 bp, synthetic construct
  source 1..17560: mol_type = other DNA
cgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattggagctccaccgcccgggctggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctaggatctgcattccaccactgctcccattcatcagttccataggttggaatctaaaatacacaaacaattagaatcagtagtttaacacattatacacttaaaaattttatatttaccttagagctttaaatctctgtaggtagtttgtccaattatgtcacaccacagaagtaaggttccttcacaaagagatcgcctgacacgatttcctgcacaggcttgagccatatactcatacatcgcatcttggccacgttttccacgggtttcaaaattaatctcaagttctacgcttaacgctttcgcctgttcccagttattaatatattcaacgctagaactcccctcagcgaagggaaggctgagcactacacgcgaagcaccatcaccgaaccttttgataaactcttccgttccgacttgctccatcaacggttcagtgagacttaaacctaactctttcttaatagtttcggcattatccacttttagtgcgagaaccttcgtcagtcctggatacgtcactttgaccacgcctccagcttttccagagagcgggttttcattatctacagagtatcccgcagcgtcgtatttattgtcggtactataaaaccctttccaatcatcgtcataatttccttgtgtaccagattttggcttttgtatacctttttgaatggaatctacataaccaggtttagtcccgtggtacgaagaaaagttttccatcacaaaagatttagaagaatcaacaacatcatcaggatccatggcacgcgcttctacaaggcgctggccgaagaggtgcgggagtttcacgccaccaagatctgcggcacgctgttgacgctgttaagcgggtcgctgcagggtcgctcggtgttcgaggccacacgcgtcaccttaatatgcgaagtggacctcggaccgcgccgccccgactgcatctgcgtgttcgaattcgccaatgacaagacgctgggcggggtttgctcgacattgggtggaaacattccaggcctgggtggagaggctttttgcttcctcttgcaaaaccacactgctcgacattgggtggaaacattccaggcctgggtggagaggctttttgcttcctcttgaaaaccacactgctcgatttgttagcagcctcgaatcaacccgggcgatcctaggcgatgagatctagctgtcgcgaagagtggcgcgcccagaggttggaggacttggaggaaatgctggttgtcccacagtatgtctgcgggggccacaggacacatattccagtgaggagtggctgatgtgtctctacacctccgaggggaagtgagaacagagtttattagttgttggatggaggtagtggaacatcctggcctgtggcgaggttcctagtgcactcgggcctcagttccagttcagacaacagagtgtttaacccttcagggtcttctgctgttctgggattcctggaagccaccagaggctatggataaagccatatggggaagcagagaggggaagaaacaagtttcttgttgcccatgaatcgggcagagggggaacctgaactaagcctgctgttttaaattggagtttcaatattgggtcatggtagcttgtgccctagtgctgggatcaggctgcactcagaaccaggaaagtgtacactgtccttatagtgtaggtattgacagctgctgagacttggggagctaaaggtagaagaggtggcttggctcccttctctaagtgcatagacaggcaaagcaacaggttcgacctatggatcccaagttatgctactaaagaagtatggcttccagaacaaagagatggtgactattccatggacactaatgggagatcctagtttctcctcctggaagcctcaggacatggacagagtttaggtgttgagcagctgctgagcagctgctgacaaaccaggcctgtctggctgacccgcagcagtagtggctgttggggtaggactgtgcttctccaggtggcctgtcaccatagcaagtgtctgggtatttctgggtgttgcctgtctacccaaacctacagggacatgagactctctggggcttagatgccctgagccaatttaagttctgctctagatctgggcaaatttaaggctgtgagttgactgctctggtcatggggaatattcaaacctttccaagcgatttaaaaaaaaaaaaaaagcatgtatgagtgtgtgtctgtgtgtacctgtgttcctgctgctcacaatgggccaaaagatggtgtctgctaccctaggactggggttaaaggtgtgagccaccatgtgggtgctgggagccaaacccagttccctctgaatgagtagcaagtactctcacctgctgaggcatctctccagcccccttcttgtgcgattttaaactttttgattttgcacttatttatctgtaatgcacgtggataagctgggcctctctctttccacgcgacgaccccctgggattgagctcaggttctcaggcttggcagtcagtgccaccctcctcgaggaactttgaacgagaggctaacttgtgcaatgctgtgcagagagatcagggaaggggcctgggactgactcacatcccagcggggcagctagagataacggtcttcctgcctttgaggatgaagatcagaagaaaatcccaggagcatgctggcattaggagcaaggaagaactttgagcaggcagagccgcctagtgacagaaaggggtgcctctggctaaagggtgtgtctgggaaggctaaacgtggcacagtgattgctcaagtcgaatgtagcatagggcctcccttatcagagagtgatgctcagctgacctttaggtttcttcccctctgctgagtgactaagggaatccacaaggcagctggagggcaatgcctgggctaggcagtgatgggatctttcccacttctctttcctacccagggacagtggcttctccagtcagggtgtagacacctacgtggagatgaggcctgtctcgacttcttcaagtgactccttctttaagcaaggtgcggaggtaccgggatgaggggagagaagggtgaatgggcctaggggccgggaactgggcctgaggaacagcaaggtgtgacagggttgccccgatgcctttgccttctacagatctggacaaagaggccagccggcccctggagctctgggacctgctccacttctccagccaagtggctcagggcatggccttccttgcttctaaaaacgtgagtctgggtgggggaaaagctgaattcctactggtcgtgggaatgaggaagaggaggcccagtgagaaagaacggaggaatagaggaatgtacagagggcctcaggaggcagcgggacgaagcagtacccagggcagactcctggaggtctagcctcagccttggtatgcctgtttctctatgtggccaccaacaggcttaacaatggtttcactcagccaagcaaactagacaacagctgcttctcctcataaaacatagtcttgttggatgagctgtaaggatgttggggctcagaccaccaccctcagagcactctagaggggcaatggtgtgacttctggctcttctctttgctatgacaatttaggttctgtaagcctcagggtctgtgtctgcaaactgggatgcatataacttcgtatagcatacattatacgaagttattatttgcaggaattgtttaggggtctaacgggttgttgtgtatgcaaatgcttgggaaagcacctggtattgtgtacttggaggtggcagctgttggtgttgtgtatgaggtgggccacaggctgctcacagcacagggactcattgcctctgaggcaggagcagccctgtggctccaagggccagagggccattggaaacagggctgtggggctagaattctaaatgttaaattgtccacatcaaatcgtctcagacctcaggcctgcacaggtttagacaggagtagacagtagactaccaaaacctgcatctacttcaacagagacccaagacctcctgctcctcctctggtcctcaggcctcagggaaggataaactgactaataatctctctgcgctttcttcagtgcatccaccgggacgtagcagctcgaaacgtgctgttgaccagcggacatgtggccaagattggggactttggactggctagggacatcatgaatgactccaactatgttgtcaagggcaatgtgagtgccgagagagagagagagagagggagggagagagagagagagagagagagagagagagagagagagagagagagagagagagagagagagagagattgagattggttgggcaggctgtggagagcccttgactgacatggtactgtcttgtcaggcccgcctgcctgtaaagtggatggccccagagagcatctttgactgcgtctacacagttcagagtgatgtgtggtcctacggcatcctcctctgggagatcttctcgcttggtgagctgctaagcctgttttcaggtgcagcctagggctgacccgtctttggctatgccatcggtgtcttgaacctcatggacgaaatctactcaaagatgtcggtgtccaagacagagcaagggctggcaggagcaatgggatggtgagggctcttgaaccagtactgcagcacctgtgtgtaattcagaccctctggctgatgtacacttcctgggtccttccttcactcgcctctgccctttacactctgcctgagaagcgtgtggctggggaagtgggaagcaagtatggtctcttatgtgatcttggactctgcaggtctgaacccctaccccggcatcctagtgaacaacaagttctacaaactggtgaaggatggataccaaatggcccagcctgtatttgcaccgaagaacatgtacgcaaaggcactcaacccaggggtgggtggttctagggttttggtagttgttggaagagtgcatggccctggtctctgtgtacaaggtgctagagccaggtttccatcttcattgcattgaggttctctacttagctttgaaccccagcggcctcaagcatgctaacaggtgctgtatcactgggccgtatctctagccctcaaacttacttttgactagtgctggggtccttcacctctccctggtgatggatgtgcccgaggaggctgacacgggcaacgcttgtcttgtttccagatacagcatcatgcagtcctgctgggacctggagcctaccagaagacccaccttccaacagatctgcttcctcctccaggagcaggcccgactggagaggagagaccaggtgagagggatggagcagatgcctggctggctagagcaggctgggccgggtctgggaaacttcagctgtttctggcttctccccaccccaggactatgctaacctgccaagcagcggtggcagcagcggcagtgacagtggtggtggcagcagcggtggcagcagcagtgagccagaagaggagagctccagtgaacacctggcctgctgtgagccaggggacatcgcccagcccctgctgcagcctaacaactaccagttctgctgatcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcgtttaaacaaccgcggcacttaagtagttaactttaaataatgccaattatttaaagttaataggcgatcgcaccatatgaatctcgaggttatgtacctgactgatgaagttcctatactttctagagaataggaacttcgaagggttccgcaagctctagtcgagccccagctggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctagagaattgatcccctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatggccgatcccatggtttagttcctcaccttgtcgtattatactatgccgatatactatgccgatgattaattgtcaacaggctgcaggtcgaaaggcccggagatgaggaagaggagaacagcgcggcagacgtgcgcttttgaagcgtgcagaatgccgggcctccggaggaccttcgggcgcccgccccgcccctgagcccgcccctgagcccgcccccggacccaccccttcccagcctctgagcccagaaagcgaaggagcaaagctgctattggccgctgccccaaaggcctacccgcttccattgctcagcggtgctgtccatctgcacgagactagtgagacgtgctacttccatttgtcacgtcctgcacgacgcgagctgcggggcgggggggaacttcctgactaggggaggagtagaaggtggcgcgaaggggccaccaaagaacggagccggttggcgcctaccggtggatgtggaatgtgtgcgaggccagaggccacttgtgtagcgccaagtgcccagcggggctgctaaagcgcatgctccagactgccttgggaaaagcgcctcccctacccggtagaatttcgacgacctgcagccaaagcgctagagccagacatgataagatacattgatgagtttggacaaaccacaactagaatgcagtgaaaaaaatgctttatttgtgaaatttgtgatgctattgctttatttgtaaccattataagctgcaataaacaagtttagctatccatcagtcgagaattggctcccacggacactccgccccttctgagataccgcatcagtgtttcaggcttcttccatgtcccttcctgcataatctcgaccatagacacctgtttctcggccatgtctattgcggcacccaccctggcggaatggccggtccaggtcctatacctccctttattgggggtggcgtcccgtttgttcagcagcacccaggcatcactgaaaatcttctccatggcaggggcggtcaggggcgtggtagtaatgcgggctttgtttgaccggtgtataggtgggaagagcacagcgtctggatgttctctaagcccactgacgtccagccagtcattgagcactgctgtggttcgccgggagagcactttgtcaaggcccgcagctgtggtgattgtcttagtgtgactgatgtgcagggtcactgtgtctcctgtctgatccaagtccccaaccctgatgcgtgatatttcagacattctcatgagggtgttataggctacgaacaggaaagccctgttgcgcaggtcgaccagccgctcgctcctgctgagcagcacatccagcagcttcaggtcgtcccagcgcagggggattgcctgtcctgtcctctcacccttttcggttgcggcttctcttctaattcttctcatggcaagactcactgacttgtcgtcggacaagggtggcagtccgcagtgggacagaagcatattaagcatagcatagtgcttgtcgatggtagtggaggccaggtctgcatcgtgcaactgaagaaagtattcgcgtgccatttcaggactaattggaaaccatgcaagctgccgagcatggcaccatctggcccagctatgaaagaccaacctcaaatccctcaaagtgttaggagcgtacgccccctggtcattcataaacctcatgaagttttcagcggcctcctgatactctttgccgatgtttcgcaggaatccaccagatgaaccactaataatcagctcagaaaccttcctcttcttcttaggcatggccgcaggaaagcagagccctgaagctcccatcaccggccaataagagccaagcctgcagtgtgacctcatagagcaatgtgccagccagcctgaccccaagggccctcaggcttgggcacactgtctctaggaccctgagagaaagacatacccatttctgcttagggccctgaggatgagcccaggggtggcttggcactgaagcaaaggacactggggctcagctggcagcaaagtgaccaggatgctgaggctttgacccagaagccagaggccagaggccaggacttctcttggtcccagtccaccctcactcagagctttaccaatgccctctggatagttgtcgggtaacggtggacgccactgattctctggccagcctaggacttcgccattccgctgattctgctcttccagccactggctgaccggttggaagtactccagcagtgccttggcatccagggcatctgagcctaccaggtccttcagtacctcctgccagggcctggagcagccagcctgcaacacctgcctgccaagcagagtgaccactgtgggcacaggggacacagggtggggcccacaacagcaccattgtccacttgtccctcactagtaaaagaactctagggttgcggggggtgggggaggtctctgtgaggctggtaagggatatttgcctggcccatggagctagcttggctggacgtaaactcctcttcagacctgaagttcctatactttctagagaataggaacttcggaattcgatatcaagctaagcttgattaactttaaataatgccaattatttaaagttagtagcgtcgcacgtgaaatcgatataacttcgtatagcatacattatacgaagttatggtaccggtaacctatttgcaggaattgtttaggggtctaacgggttgttgtgtatgcaaatgcttgggaaagcacctggtattgtgtacttggaggtggcagctgttggtgttgtgtatgaggtgggccacaggctgctcacagcacagggactcattgcctctgaggcaggagcagccctgtggctccaagggccagagggccattggaaacagggctgtggggctagaattctaaatgttaaattgtccacatcaaatcgtctcagacctcaggcctgcacaggtttagacaggagtagacagtagactaccaaaacctgcatctacttcaacagagacccaagacctcctgctcctcctctggtcctcaggcctcagggaaggataaactgactaataatctctctgcgctttcttcagtgcatccaccgggacgtagcagctcgaaacgtgctgttgaccagcggacatgtggccaagattggggactttggactggctagggacatcatgaatgactccaactatgttgtcaagggcaatgtgagtgccgagagagagagagagagagggagggagagagagagagagagagagagagagagagagagagagagagagagagagagagagagagagagagattgagattggttgggcaggctgtggagagcccttgactgacatggtactgtcttgtcaggcccgcctgcctgtaaagtggatggccccagagagcatctttgactgcgtctacacagttcagagtgatgtgtggtcctacggcatcctcctctgggagatcttctcgcttggtgagctgctaagcctgttttcaggtgcagcctagggctgacccgtctttggctatgccatcggtgtcttgaacctcatggacgaaatctactcaaagatgtcggtgtccaagacagagcaagggctggcaggagcaatgggatggtgagggctcttgaaccagtactgcagcacctgtgtgtaattcagaccctctggctgatgtacacttcctgggtccttccttcactcgcctctgccctttacactctgcctgagaagcgtgtggctggggaagtgggaagcaagtatggtctcttatgtgatcttggactctgcaggtctgaacacctaccccggcatcctagtgaacaacaagttctacaaactggtgaaggatggataccaaatggcccagcctgtatttgcaccgaagaacatgtacgcaaaggcactcaacccaggggtgggtggttctagggttttggtagttgttggaagagtgcatggccctggtctctgtgtacaaggtgctagagccaggtttccatcttcattgcattgaggttctctacttagctttgaaccccagcggcctcaagcatgctaacaggtgctgtatcactgggccgtatctctagccctcaaacttacttttgactagtgctggggtccttcacctctccctggtgatggatgtgcccgaggaggctgacacgggcaacgcttgtcttgtttccagatacagcatcatgcagtcctgctgggacctggagcctaccagaagacccaccttccaacagatctgcttcctcctccaggagcaggcccgactggagaggagagaccaggtgagagggatggagcagatgcctggctggctagagcaggctgggccgggtctgggaaacttcagctgtttctggcttctccccaccccaggactatgctaacctgccaagcagcggtggcagcagcggcagtgacagtggtggtggcagcagcggtggcagcagcagtgagccagaagaggagagctccagtgaacacctggcctgctgtgagccaggggacatcgcccagcccctgctgcagcctaacaactaccagttctgctgaagtgggagggagagccgagtcctgccgctctctacgtcccagcttggcctcctccatggcacgggcgacatggggagaacatatggacttcgccctcagcttggcccagctctgacacttcagaacatgaggggtctggggaggtcagaggccccgtttgttcccagagcctgggccatcactgccagtggggttctcacagtgctagcctctatatttactatgccaactggtgcacccctagttctctttctccatcctattcccattttaaaaaacccgtcccaaactctcgtgtttcaatggaaagactgatttatgtctcaaaagacaagagtctcaaaggctgtgggtaagctgaaggcttgcctccctgacagatgcttagactacaggcttcttgggacaggtggccccttcctaagctcacaggagtggccaccactcttgaccttcactctgtctatagtcccgcctcatcctggatcttgtactgagcggcagctaaaagtgttctacccagtgccctgtcactctagactggaaggtatggggcctgatgcaaggctgaccacaccaacaaacaccgtgtgctcctctccaagctgactcgtcctcattaactgtcaacattaaactaacagcattaacacagccagcagggtctggttctttgcagcacacaggtgccttcaagctccctaggaccgacctccttccacctattcctcctgacaagggaccaacaagcacccacttccagagccaagggacacagcattgaacaggctcttgcttcatcaaaaccagataatttaataaactttattcttgttgggggaagaacgagagggaaagccacctatcccaccctctcaataaattaaatagcacttggccaatcctgggccctaagcaaaccgcgggcacctgagcccatgctgctgtttgcttatggccctatctgcttccccttcacagaagggagttttcctactgcttgtcagctatgtcacaaccacgggccacatcccctctgtggtgagggtctgaggacccagaggacgtagggtatagtagcagtggccagagaaaggaggaaggattctcttcccccaaacccggggctgagagagtcctggaggagctgttccttctcctcaactgccccaaggtgtgggaacagggagagttggaggaacagagaatgcacccctcctttagggagaatgaagggccccaagggattccaagagtaaataagccagcaaggcaggctaggaagcccacaagacaggagccagagctcctccccgcgggacagcttagctcaatggcggctccacctggtgcaggaacccgggtggttgtgggtctgggcaaccaggccccggccttcctatcagcaccagatccttaagaacatcatgtcctgcctccacgtgctctctcaggtcaaggagaggcttcccagcgcctctcctgagccccagaggtcgaggccccactgtgctcatgaaatggaagctctttggtcagttccagcagtcagaggccctccctaaagaggactgcaggtccctcaataggactgccctgggcaagggccagattatggcttgagagagatgatgcaggccctgtacataaaaacctggcttattccaccactgaggctacttcctcttcctctccttcctcctgggcctccgtcttgatctgggcaaccccaaggcgatagagggtgtcgcgaatgaccacctcgccaggctggcagctctgcacaatgtcatggatctgcctgttgacaatctctcggctggtgaggaagtccatgaggcggttgtagaggtaatagtgggtggcattgtcaaaggcgatgggcctctggcggacactgctgagcttgctgtccgtggtagaggcataaggtgccagctctgggcacagtgataaggaacggtggccagcactggggtcactgggatcgggctttgtgtttgcatggacaatacgccgtgtcgcactcttggtgactggatgctgcactgagttttcagcctctgtcacatccacagaataatgctggtccaagagttcagggcaggacacactctacaagagagaagagaagctgctctgtaagagggatgactgagggagaaccttgctggagagagcagaagccaggcagccccgtttaaatcctaaaggctgtgtacactgtgtctcctgtgccccatttcttcatgtaagaatgggcataggaaaatggtccccccagcacaggtgctggactgtttgagagctataccatcactgtacctggacaatgtgggcaccagaggaataggaggatctggctcggtgggacacttcagacacttttgggtgcggccgcgtaccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaa

SEQ ID NO: 6 - DNA, 23 bp, synthetic construct
  source 1..23: mol_type = other DNA
ggctggtaagggatatttgcctg

SEQ ID NO: 7 - DNA, 25 bp, synthetic construct
  source 1..25: mol_type = other DNA
tcatgctccaagaaattgtggtaga

SEQ ID NO: 8 - DNA, 25 bp, synthetic construct
  source 1..25: mol_type = other DNA
gctgcttctcctcataaaacatagt

SEQ ID NO: 9 - DNA, 25 bp, synthetic construct
  source 1..25: mol_type = other DNA
atttgcatacacaacaacccgttag

SEQ ID NO: 10 - DNA, 22 bp, synthetic construct
  source 1..22: mol_type = other DNA
cttggctggacgtaaactcctc

SEQ ID NO: 11 - DNA, 24 bp, synthetic construct
  source 1..24: mol_type = other DNA
aagtacacaataccaggtgctttc

SEQ ID NO: 12 - DNA, 20 bp, synthetic construct
  source 1..20: mol_type = other DNA
aaggcgatagaaggcgatgc

SEQ ID NO: 13 - DNA, 20 bp, synthetic construct
  source 1..20: mol_type = other DNA
tcatctcaccttgctcctgc

(19) United States
(12) Patent Application Publication
(10) Pub. No.: US 2024/0206441 A1
(43) Pub. Date: Jun. 27, 2024

(54) Method for Constructing Murine Model with Mutations in Csf1r Gene and Application thereof

(71) Applicants: Beijing Tiantan Hospital, Capital Medical University, Beijing (CN); Yangzhou University, Yangzhou (CN)

(72) Inventors: Jun XU, Beijing (CN); Xiaohong WANG, Yangzhou (CN); Yanli WANG, Beijing (CN); Jiwei JIANG, Beijing (CN); Linlin WANG, Beijing (CN); Shiyi YANG, Beijing (CN)

(21) Appl. No.: 18/545,693

(22) Filed: Dec. 19, 2023

(30) Foreign Application Priority Data
Dec. 23, 2022 (CN) 202211663356.4

(51) Int. Cl. A01K 67/0276; A61K 49/00; C07K 14/715; C12N 15/85

(52) U.S. Cl. CPC A01K 67/0276; A61K 49/0008; C07K 14/7153; C12N 15/8509; A01K 2217/054; A01K 2227/105; A01K 2267/0356; C12N 2015/8527

(57) ABSTRACT

A mutated Csf1r gene is disclosed. The mutated Csf1r gene is obtained by changing the 2557th nucleotide of a Csf1r gene from C to A, leading to obtain a mutated protein encoded by the mutated Csf1r gene substitute the 853rd amino acid from proline to threonine. The mutated protein, expression vectors, recombinant viruses, recombinant cells, recombinant bacteria, or recombinant vectors are also disclosed. A method for constructing a murine model with mutations in Csf1r gene. This method includes introducing the targeted vector containing the mutated Csf1r gene into mouse embryonic stem cells, followed by injection into blastocysts to generate F0 generation mice. The F0 generation mice are then bred with mice that specifically express Cre enzyme in tissues, followed by screening. The constructed murine model has significant applications in studying the pathogenic mechanisms of brain diseases caused by microglial cell dysfunction and screening valuable medicine for treating brain diseases.

### CROSS REFERENCE OF RELATED APPLICATIONS

The present application claims priority of Chinse Patent Application No. 202211663356.4, filed on Dec. 23, 2022, entitled “Method for Constructing Murine Model with Mutations in Csf1r Gene, and Application thereof,” in the China National Intellectual Property Administration (CNIPA), the entire contents of which are hereby incorporated by reference in their entireties.

### REFERENCE TO AN ELECTRONIC SEQUENCE LUSTING

The contents of the electronic sequence listing (11-SEQ.xml; Size: 37,825 bytes; and Date of Creation: Dec. 20, 2023) is herein incorporated by reference in its entirety.

### FIELD OF THE DISCLOSURE

The disclosure relates to the field of animal model construction techniques, and especially to a method for constructing a murine model with mutations in Csf1r (Colony Stimulating Factor 1 Receptor) gene, and applications thereof.

### BACKGROUND OF THE DISCLOSURE

Functional impairment of microglia resulting from mutations in the Csf1r gene is a rare leukodystrophy. Clinically, it often manifests as rapidly progressive cognitive dysfunction, parkinsonian-like motor impairment, and psychiatric behavioral abnormalities. Imaging reveals conspicuous alterations in brain white matter, ventricular enlargement, brain atrophy, dysgenesis of the corpus callosum, multiple scattered calcifications and so on. Pathological anatomy findings include characteristic axonal spheroid accompanied by pigmented glial, diffuse axonal degeneration and demyelination. The disease was formerly known by various names such as hereditary diffuse leukoencephalopathy with spheroids (HDLS), adult-onset leukodystrophy with neuroaxonal spheroids and pigmented glia (ALSP), and Csf1r-related encephalopathy, all based on these distinctive phenotypic features.

In the human genome, the Csf1r gene is located on the q32 region of the long arm of chromosome 5, and in mice the Csf1r gene is located on chromosome 18. The Gene ID of the Csf1r is 12978 in NCBI. The Csf1r protein is a receptor protein primarily distributed on the cell surface, comprising five functional domains. The five functional domains include 5 immunoglobulin-like motifs in the extracellular domain, a transmembrane domain, a juxtamembrane domain, and two tyrosine kinase domains. As of September 2022, a total of 126 pathogenic mutation sites have been reported worldwide, with approximately 90% located within the two tyrosine kinase domains. In vitro studies have identified two possible mechanisms by which Csf1r mutations lead to impaired autophosphorylation function: one involving a negative regulatory mechanism, mutations within the juxtamembrane domain or the kinase insert region result in reduced kinase activity, inhibiting downstream target phosphorylation; the other involving a loss-of-function mechanism, mutations within the tyrosine kinase domains lead to kinase inactivation, rendering it incapable of further signal transduction. However, the mechanisms in vivo remain unclear. Previously, a missense mutation c.2563C>A (p.P855T) in exon 20 of the Csf1r gene was initially identified in a family with hereditary diffuse leukoencephalopathy with spheroids (HDLS). Through validation within the family and research involving conditional knockout mouse models, it has been confirmed that the c.2563C>A point mutation in the Csf1r gene is a pathogenic site causing brain diseases associated with microglial dysfunction.

### SUMMARY OF THE DISCLOSURE

The first object of the present invention is to provide a mutated Csf1r gene, mutated protein, expression cassette, recombinant virus, recombinant cell, recombinant bacterium, or recombinant vector.

The second object of the present invention is to provide a method for constructing a murine model with mutations in the Csf1r gene.

The third object of the present invention is to provide an application of the method for constructing a mutated Csf1r gene and a murine model with Csf1r gene mutation in the preparation of medicine for the treatment of brain diseases caused by microglial dysfunction.

The present invention provides a mutated Csf1r gene, where the 2557th nucleotide of the Csf1r gene has mutated from C to A, as shown in SEQ ID NO: 1.

The present invention further provides a mutated Csf1r protein encoded by the aforementioned mutated gene. The mutated Csf1r protein features a substitution of proline with threonine at the 853rd amino acid position of the Csf1r protein. The amino acid sequence of the mutated Csf1r protein is shown in SEQ ID NO: 2.

The present invention further provides expression cassettes, recombinant viruses, recombinant cells, recombinant bacteria, or recombinant vectors containing the mutated Csf1r gene.

The present invention further provides a method for constructing a murine model with mutations in the Csf1r gene, comprising the following steps:
(1) constructing a targeting vector, Csf1r^{P853T/+}, containing the mutated Csf1r gene;
(2) electroporating the targeting vector Csf1r^{P853T/+} obtained in step (1) into mouse embryonic stem cells and verifying positive clones.
(3) injecting the verified positive mouse embryonic stem cells from step (2) into blastocysts and transplanting the blastocysts into the uteri of female mice to obtain F0 generation mice.
(4) breeding the F0 generation mice obtained in step (3) with mice that express Cre enzyme in a tissue-specific manner and selecting F1 generation heterozygous mice that carry both the point mutation described in step (1) and the Cre gene in their genomes.
(5) pairing any two heterozygous F1 generation mice obtained in step (4) and selecting F2 generation homozygous mice, thereby obtaining the desired murine model.

Preferably, the mice that express Cre enzyme in a tissue-specific manner are mice that express Cre enzyme in macrophages.

Preferably, the F2 generation homozygous mice are mice afflicted with brain diseases caused by microglial dysfunction.

The present invention further provides an application of any of the mutated Csf1r gene, mutated Csf1r protein, expression cassette, recombinant virus, recombinant cell, recombinant bacterium, or recombinant vector, and the construction method in the preparation of medicine for the treatment of brain diseases caused by microglial dysfunction.

Advantages: Compared to the prior art, the present invention offers the following significant benefits: (1) In contrast to other genetically modified or knockout mouse models of microglial encephalopathy, the present invention can simulate a broader range of psychiatric symptoms in addition to cognitive impairments, including the manifestation of both manic and depressive symptoms, as well as schizophrenia-like symptoms. (2) The present invention can replicate peripheral symptoms of clinical microglial encephalopathy, such as skeletal developmental anomalies, splenic structural damage and hyperfunction, systemic inflammation, ascites, colonic ulcerative inflammation, and other symptoms. (3) The present invention allows for the observation of the characteristic pathological structure of microglial encephalopathy—axonal spheroids. (4) These mice have a longer lifespan, making it convenient for longitudinal observation of changes in behavior, pathology, and gene expression levels at different levels. (5) There are significant differences in pathology and behavior between female and male mice.

### BRIEF DESCRIPTION OF THE DRAWINGS

FIG. 1A is a schematic diagram showing the wild-type Csf1r gene.

FIG. 1B is a schematic diagram showing the linear vector for constructing Csf1r^{P853T/+}, where the triangle symbols indicate LoxP sites, the diamond symbols indicate SDA (self-deletion anchor) sites, and the asterisk symbols represent the point mutation sites.

FIG. 2 is a schematic diagram showing the PCR primer design sites.

FIG. 3 shows the results of positive clone PCR screening at the 3′ arm end.

FIG. 4 shows the results of positive clone PCR screening at the far-end LoxP site.

FIG. 5 shows the results of positive clone PCR screening at the Neo site.

FIG. 6 shows the design of the Southern blot detection region.

FIG. 7 shows the results of Southern blot detection.

FIG. 8 shows the sequencing results of the Csf1r gene point mutation, changing from CCC to ACC.

FIG. 9A shows the weight gain graph for C57/BL6, Csf1r^{P853T/+} type I, and Csf1r^{P853T/+} type II mice.

FIG. 9B shows the phenotypes of Csf1r^{P853T/+} type I and Csf1r^{P853T/+} type II mice.

FIG. 10A is a schematic diagram of the three-chamber social test.

FIG. 10B shows the results of the three-chamber social test for C57/BL6, Csf1r^{P853T/+} type I, and Csf1r^{P853T/+} type II mice.

FIG. 11A is a schematic diagram of the Y-maze.

FIG. 11B shows the results of the Y-maze test for C57/BL6, Csf1r^{P853T/+} type I, and Csf1r^{P853T/+} type II mice.

FIG. 12A shows the results of the open-field test for C57/BL6, Csf1r^{P853T/+} type I, and Csf1r^{P853T/+} type II mice.

FIG. 12B shows the route map of the open-field test for C57/BL6, Csf1r^{P853T/+} type I, and Csf1r^{P853T/+} type II mice.

FIG. 13A shows the nest-building test for C57/BL6, Csf1r^{P853T/+} type I, and Csf1r^{P853T/+} type II mice.

FIG. 13B shows the nest-building results for C57/BL6, Csf1r^{P853T/+} type I, and Csf1r^{P853T/+} type II mice.

FIG. 14A shows the rotarod test.

FIG. 14B shows the results of the rotarod test for C57/BL6, Csf1r^{P853T/+} type I, and Csf1r^{P853T/+} type II mice.

FIG. 15A shows the stride calculation pattern.

FIG. 15B shows the results of the gait test for C57/BL6, Csf1r^{P853T/+} type I, and Csf1r^{P853T/+} type II mice.

FIG. 16A shows the PPI (pre-pulse inhibition) test.

FIG. 16B shows the results of the PPI test for C57/BL6, and Csf1r^{P853T/+} type I mice.

FIG. 17 shows HE (Hematoxylin and Eosin) staining of the cortex in for C57/BL6 and Csf1r^{P853T/+} mice, with arrowheads pointing to axonal spheroid-like changes.

FIG. 18 shows HE staining of the corpus callosum in C57/BL6 and Csf1r^{P853T/+} mice.

FIG. 19 shows LFB (Luxol Fast Blue) staining of the cortex in C57/BL6 and Csf1r^{P853T/+} mice.

FIG. 20 shows MRI-T2 phase imaging of C57/BL6 and Csf1r^{P853T/+} mice.

FIG. 21 shows the immunohistochemical results of CD68 staining in the cortex region of C57/BL6 and Csf1r^{P853T/+} mice.

FIG. 22 shows the expression of Csf1r in the spleen and brainstem of C57/BL6, and Csf1r^{P853T/+} mice.

### DETAILED DESCRIPTION OF THE DISCLOSURE

The following description is provided in conjunction with the accompanying figures to further elucidate the technical approach of the present invention.

### Example 1: Construction of a Murine Model with Csf1r Gene Mutations

### Step 1, Constructing the Vector

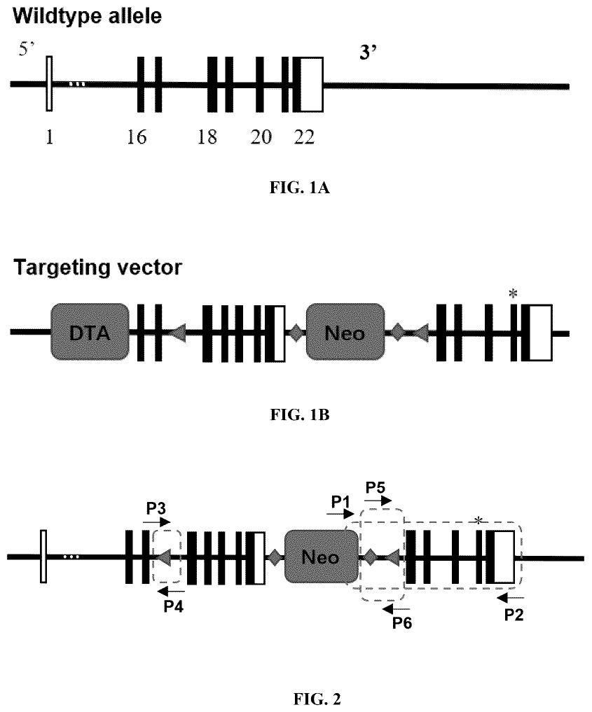

As shown in FIG. 1A, the Csf1r gene sequence, shown in SEQ ID NO: 3, is located on mouse chromosome 18 and comprises 22 exons. The start codon ATG is situated in exon 2, while the stop codon TGA is located in exon 22. The amino acid sequence encoded by the Csf1r gene is shown in SEQ ID NO: 4. The mutation site involves the change of the 2557th nucleotide of the Csf1r gene from C to A, resulting in the mutated Csf1r gene shown in SEQ ID NO: 1. The mutation also leads to the substitution of proline with threonine at the 853rd amino acid position of the Csf1r protein, giving rise to the mutated Csf1r protein with the amino acid sequence shown in SEQ ID NO: 2.

The mutation introduced at the 853rd amino acid position encoded by the Csf1r gene corresponds to the 2557th nucleotide position in the cDNA. Primers were designed to introduce the P853T (CCC to ACC) mutation into exon 20.

The construction of the vector, as shown in FIG. 1B, involves the Csf1r^{P853T/+} targeting vector, which is a linearized vector containing the P853T (CCC to ACC) mutation site. The vector also includes common elements such as DTA, loxP, sdNeo cassette, SDA, and others. The sequence of the Csf1r^{P853T/+} targeting vector is shown in SEQ ID NO: 5. This vector was constructed by Saiye (Guangzhou) Biotechnology Co., Ltd.

### Step 2, Electroporation

C57BL/6N ES cells were resuscitated and passaged using serum-free mouse embryonic stem cell culture medium (OriCell, MUXES-90061, Saiye (Guangzhou) Biotechnology Co., Ltd.). Approximately 1×10⁷ cells were counted and resuspended in electroporation buffer. To this cell suspension, 35 μg of the linearized Csf1r^{P853T/+} targeting vector obtained in Step 1 was added and thoroughly mixed. The mixture was then allowed to sit on ice for 5 minutes. The cell suspension was transferred to an electroporation cup, and electroporation was performed using the following parameters: 250V, 500 μF, and a single pulse. After electroporation, the cells were transferred to culture dishes pre-seeded with G418-resistant MEF cells. Subsequent culture was carried out using serum-containing mouse embryonic stem cell culture medium (OriCell, MUXES-90011, Saiye (Guangzhou) Biotechnology Co., Ltd.).

After 24 hours, selection was initiated by adding G418 (final concentration 200 μg/mL) to the serum-containing mouse embryonic stem cell complete medium (OriCell, MUXES-90061, Saiye (Guangzhou) Biotechnology Co., Ltd.). Over a period of 7 days, daily observation and medium changes were performed. Following the completion of drug selection, surviving clones were picked and transferred to a 96-well plate for further passaging and culture.

### Step 3, PCR Screening:

PCR amplification and electrophoresis were used to screen the clones obtained in Step 2. The primer design sites are shown in FIG. 2, and their specific sequences are provided in the table below:

TABLE 1
Primer Design

| | | | The anticipated length | |
|---|---|---|---|---|
| | | | of the PCR product | |
| | | | Wildtype | Targeting |
| Region | | Sequence | allele | allele |
| 3′arm | Neo-F1 | 5′-GGCTGGTAAGGGATA | N.A. | ~4.6 kb |
| | (P1) | TTTGCCTG-3′ | | |
| | 3′arm-R | 5′-TCATGCTCCAAGAAA | | |
| | (P2) | TTGTGGTAGA-3′ | | |
| loxP | loxP-F | 5′-GCTGCTTCTCCTCATA | 219 bp | 259 bp |
| site | (P3) | AAACATAGT-3′ | | |
| | loxP-R | 5′-ATTTGCATACACAAC | | |
| | (P4) | AACCCGTTAG-3′ | | |
| Neo | Neo-F2 | 5′-CTTGGCTGGACGTAA | N.A. | 273 bp |
| site | (P5) | ACTCCTC-3′ | | |
| | Neo-R | 5′-AAGTACACAATACCA | | |
| | (P6) | GGTGCTTTC-3′ | | |

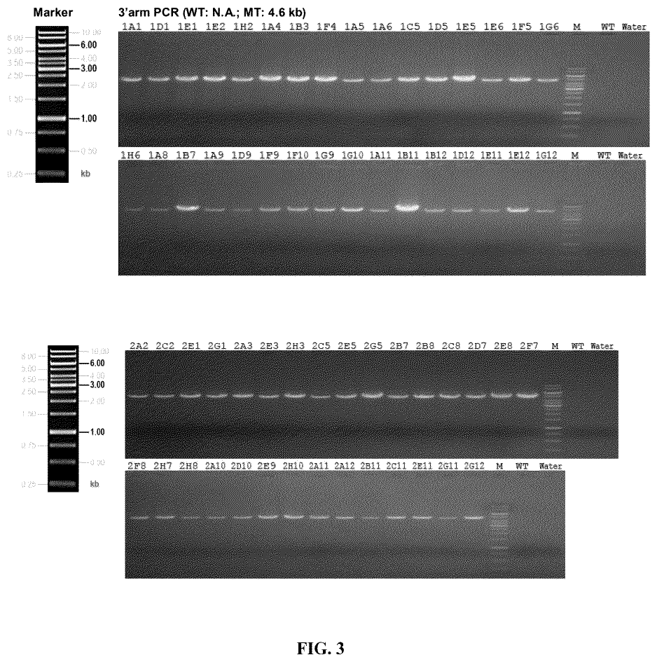

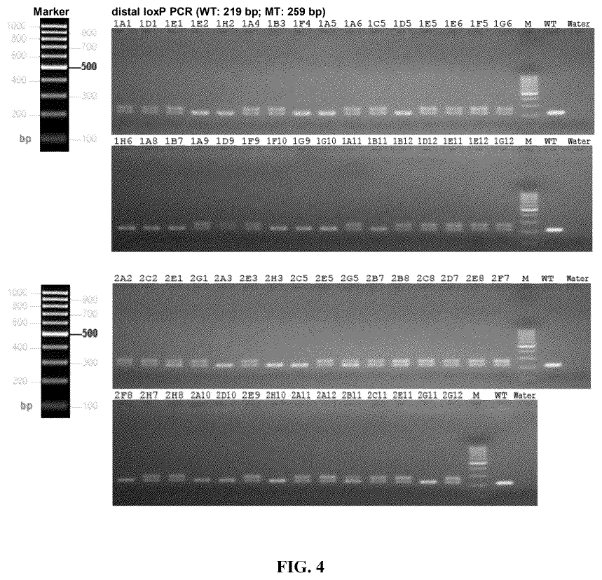

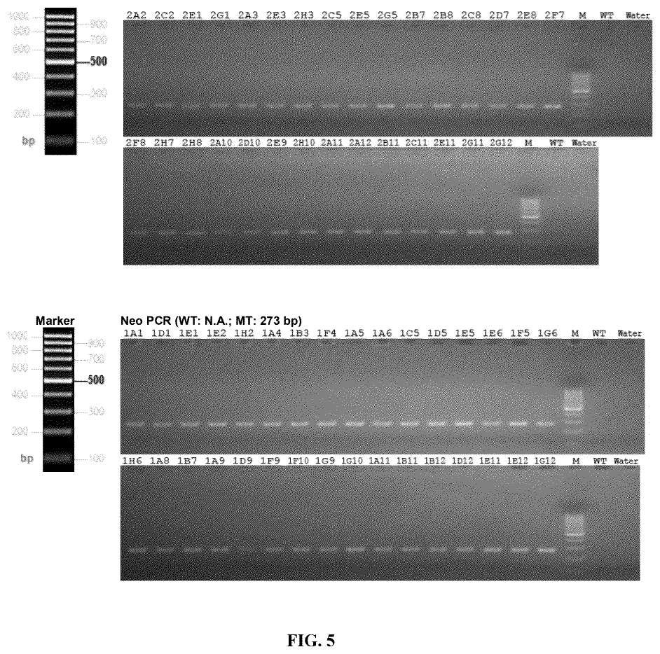

The results of the 3′arm end positive clone PCR screening are shown in FIG. 3, the results of the far-end loxP site positive clone PCR screening are shown in FIG. 4, and the results of the Neo site positive clone PCR screening are shown in FIG. 5. Samples 1A1, 1D1, 1E1, 1A4, 1B3, 1A6, 1C5, 1E5, 1E6, 1F5, 1G6, 1A9, 1D9, 1F9, 1A11, 1B12, 1D12, 1E11, 1E12, 1G12, 2A2, 2C2, 2E1, 2G1, 2E3, 2E5, 2G5, 2B7, 2B8, 2C8, 2D7, 2E8, 2F7, 2H7, 2H8, 2E9, 2A1, 2A12, 2B11, 2C11, 2E11, and 2G12 are sequenced. Among these samples, 1C5, 1F5, 1A9, 1E11, 1E12, 2C2, 2E3, 2G5, 2E8, 2H7, 2A11, 2A12, 2C11, and 2E11 were confirmed as potential target ES clones through sequencing.

### Step 4, Southern Blot for Positive ES Cell Selection

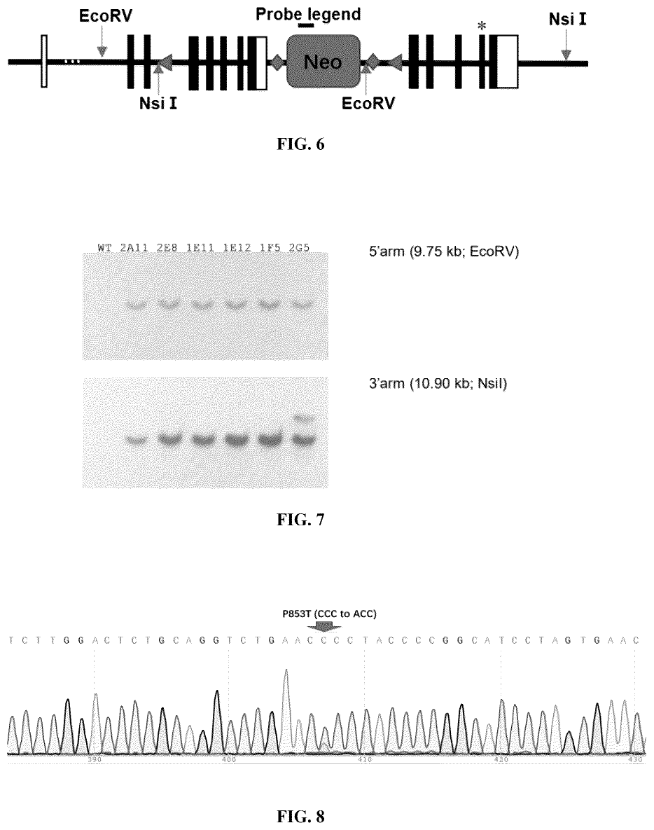

Positive clones (2A11, 2E8, 1E11, 1E12, 1F5, and 2G5) identified through PCR screening were further amplified and subjected to Southern blot analysis for confirmation. The Southern blot detection region is outlined in FIG. 6. Genomic DNA was digested using either EcoRV or NsiI, and hybridized with a Neo probe. The sequences of the Neo probe primers are as follows:

Neo-F1:
AAGGCGATAGAAGGCGATGC;
Neo-R1:
TCATCTCACCTTGCTCCTGC.

In Southern analysis, the Neo probe can detect the following DNA fragments from the target allele: ˜9.75 kb (digested with EcoRV) and ˜10.90 kb (digested with NsiI). Among the six ES clones, five (2A11, 2E8, 1E11, 1E12, and 1F5) were confirmed as positive through Southern blot detection, as shown in FIG. 7.

### Step 5, Mouse Breeding

ES cells were injected into embryos, and the embryos were then transplanted into the uteri of surrogate mother mice. The surrogate mother mice gave birth to the F0 generation of genetically engineered mice, known as Knockout-floxed mice. After reaching 8 weeks of age, the Knockout-floxed mice were housed together with 8-week-old mice that expressed Cre enzyme specifically in macrophages (Saiye (Suzhou) Biotechnology Co., Ltd.). This breeding resulted in the F1 generation of heterozygous Csf1r^{P853T/+} mice. A pair of F1 generation mice were selected for mating to obtain the F2 generation of homozygous Csf1r^{P853T/+} mice. Sequencing was performed to confirm the presence of the single-base mutation at the 2557th nucleotide position of the Csf1r gene cDNA, as shown in FIG. 8. This mutation corresponds to the designed mutation site, confirming the successful generation of F2 generation homozygous Csf1r^{P853T/+} mice with macrophage-specific expression of the mutated Csf1r gene.

### Example 2: Cognitive Assessment of Csf1r^{P853T/+} Mice

Experimental Materials: Csf1r^{P853T/+} mice constructed in Example 1, aged 8 months, weighing between 25-28 g, ad libitum feeding; C57/BL6 wild-type mice (sourced from the Comparative Medicine Center at Yangzhou University), aged 8 months, weighing between 25-28 g, ad libitum feeding.

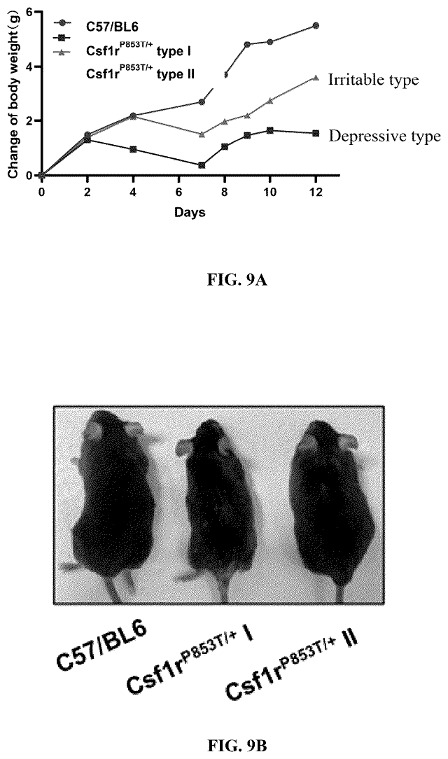

As illustrated in FIG. 9B, in comparison to C57/BL6 mice, Csf1r^{P853T/+} mice exhibit erect, shedding, and increasingly gray fur, coupled with a diminished mental state. Notably, they manifest two distinctive psychological phenotypes, encompassing a depressive type (type I) and an irritable type (type II). Furthermore, as shown in FIG. 9A, both type I and type II mice exhibit reduced weight gain in contrast to their wild-type counterparts, with type I displaying the slowest growth rates (P<0.05).”

### 1. Three-Chamber Social Test

The foundation of the three-chamber social test lies in the innate sociability of normal mice. When faced with conspecifics, they exhibit a preference for social interaction over isolation. Furthermore, when faced with unfamiliar conspecifics, normal mice have the ability to distinguish them from familiar companions and tend to engage in more interactions with the stranger ones, demonstrating a social novelty response.

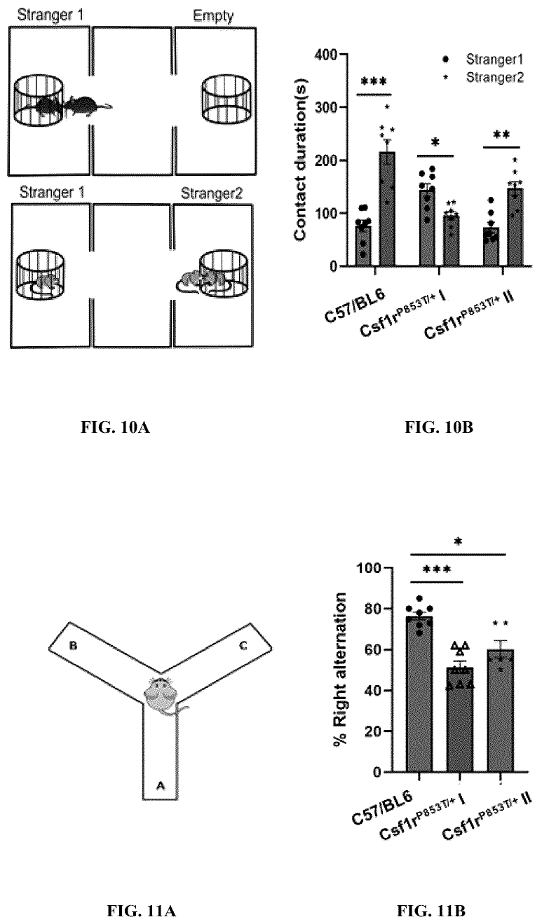

As shown in FIG. 10A, the three-chamber test involves dividing three chambers using transparent partitions. A Csf1r^{P853T/+} mouse is placed in the central chamber and allowed to acclimate to the environment for 5 minutes.

First Phase: The partition is removed, and a cage containing an unfamiliar C57/BL6 mouse (stranger1) is placed in the left chamber, while an empty cage is placed in the right chamber. A 10-minute timer is started, and the time Csf1r^{P853T/+} mouse spends in each chamber (with all four limbs inside as the criteria) is recorded.

Second Phase: The empty cage in the right chamber is replaced with another unfamiliar C57/BL6 mouse (stranger2), and once again, the time and frequency of entry by the Csf1r^{P853T/+} mouse into the right chamber are recorded. After testing one mouse, the apparatus is cleaned with 10% alcohol.

The test is repeated three times, and the same test setup is also prepared for C57/BL6 mice as a control.

### 2. Maze Test

As illustrated in FIG. 11A, a Y-maze is consisted of three arms: the novel arm, the start arm, and the other arm. Novel Arm: The novel arm is blocked off by partitions in the first phase of the test, and is opened in the second phase (i.e., the testing period). Start Arm: The start arm is the arm where the mouse is placed at the beginning of the maze test. Throughout the entire test, both the start arm and the other arm remain open.

First Phase: This is the training period. The novel arm is blocked off with partitions, and the Csf1r^{P853T/+} mouse is introduced into the maze from the start arm. The mouse is allowed to freely explore both the start arm and the other arm for 10 minutes. The next phase begins 1 hour later.

Second Phase: This is the testing period. The partition blocking the novel arm is removed, and the Csf1r^{P853T/+} mouse is placed in the maze from the start arm. The mouse is given 5 minutes to freely explore all three arms. The time spent in each arm and the number of shuttle movements within the 5-minute period are recorded. Each consecutive entry into all three arms of the Y-maze is counted as one shuttle.

The test is repeated three times, and the same test setup is also prepared for C57/BL6 mice as a control.

### 3. Nest-Building Test

On the testing day in the evening, clean cotton of the same mass is provided to the Csf1r^{P853T/+} mice using sterile forceps. After 24 hours, photographs are taken to record the condition of nest-building, and a scoring system for the nest-building is employed for evaluation, as follows:
5 points: Over 90% of the cotton pieces are torn into shreds, forming a complete nest with edges higher than half the height of a curled-up mouse;
4 points: Over 90% of the cotton pieces are torn, creating a flat nest with edges not exceeding half the height of a curled-up mouse;
3 points: 50%-90% of the cotton pieces are torn, but no nest is formed; cotton pieces are scattered throughout the cage;
2 points: 50%-90% of the cotton pieces remain untorn and intact;
1 point: Over 90% of the cotton pieces remain untouched and undamaged.

The test is repeated three times, and the same test setup is also prepared for C57/BL6 mice as a control.

### 4. Prepulse Inhibition Test

As illustrated in FIG. 16A, Csf1r^{P853T/+} mice are acclimated for 5 minutes in a testing chamber with a background noise level of 62 dB to reduce interference from background noise. Subsequently, the following prepulse stimuli are administered:

Block 1: Ten shock stimuli at 120 dB.

Block 2 includes the following six modes:
(1) Background noise at 62 dB only.
(2) Shock stimulus at 120 dB only.
(3) Prepulse stimuli at 74 dB, 78 dB, or 86 dB only.
(4)-(6) Prepulse stimuli at 74 dB, 78 dB, and 86 dB, followed by a 100 ms interval and a 120 dB shock stimulus. A total of 40 shock stimuli are presented to assess the mouse's PPI strength. These six modes are presented in a pseudo-randomized order, ensuring that the prepulse-only mode (Mode (3)) occurs once every 6 trials with intervals of 10-20 seconds between adjacent modes.

Block 3 is similar to Block 1 and serves as an adaptive contrast to analyze whether the mice exhibit adaptation during the test. After collecting the startle response amplitude data, Shanghai Xinruan VisuStartle Startle Reflex Test Software is employed for data processing and analysis. Prepulse Inhibition Efficiency (PPI %) is used to represent the strength of PPI, calculated as follows: PPI %=(startle response amplitude to shock stimulus−startle response amplitude to prepulse combined with shock stimulus)÷startle response amplitude to shock stimulus×100%. The startle response amplitude to shock stimulus is the mean response amplitude induced under shock stimulus conditions in Block 1, while the startle response amplitude to prepulse combined with shock stimulus is the mean response amplitude induced under certain prepulse intensity combined with shock stimulus conditions in Block 2.

The test is repeated three times, and the same test setup is also prepared for C57/BL6 mice as a control.

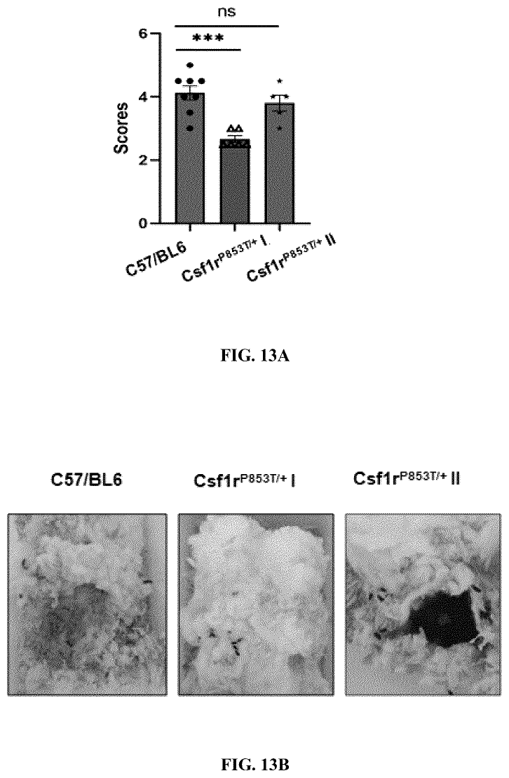

As shown in FIG. 10B, results from the Three-Chamber Social Test indicate that Csf1r^{P853T/+} type I mice spent more time exploring stranger1, but significantly less time exploring stranger2, with the time spent exploring stranger2 being significantly lower than that spent exploring stranger1. Csf1r^{P853T/+} type II mice spent significantly less time exploring stranger1 than stranger2. In FIG. 11B, results from the Y-Maze Test demonstrate that both Csf1r^{P853T/+} type I and type II mice had significantly reduced instances of correct exploration compared to wild-type C57/BL6 mice. As shown in FIGS. 13A and 13B, results from the Nest-Building Test reveal that Csf1r^{P853T/+} type I mice exhibited a significantly reduced nest-building score, while Csf1r^{P853T/+} type II mice did not show a significant reduction. In FIG. 10B, results from the Prepulse Inhibition Test indicate that Csf1r^{P853T/+} type II mice had a significantly reduced prepulse inhibition rate compared to wild-type C57/BL6 mice.

### Example 3: Motor Function Assessment of Csf1r^{P853T/+} Mice

### 1. Open Field Test

Csf1r^{P853T/+} mice are gently removed from their housing cages and placed quickly into an open field arena measuring 50×50×25 centimeters. During placement, care is taken to ensure that all mice are oriented in the same direction within the arena. The tracking system is then activated to automatically record the mice's movement distance, resting time, and active time over a 15-minute test period.

The test is repeated three times, and the same test setup is also prepared for C57/BL6 mice as a control.

### 2. Rotarod Test

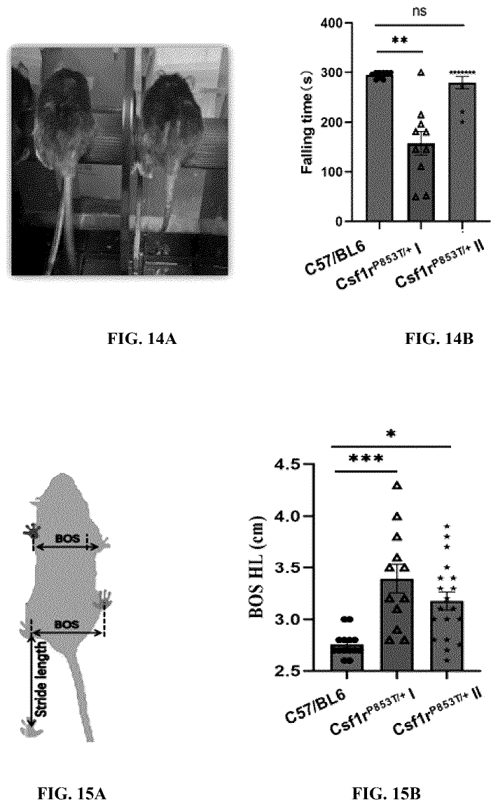

As illustrated in FIG. 14A, the rotarod apparatus is prepared with the rotation speed set and power supply activated, causing the rod to rotate automatically. Csf1r^{P853T/+} mice are placed on the rotarod instrument with a constant rotation speed of 15 revolutions per minute (r/min) for continuous training over three days. On the fourth day, three consecutive trials are conducted, each lasting for 5 minutes. A 30-minute rest period is observed between each trial, and the time spent by the mice on the rotarod during the three trials on the fourth day is recorded for analysis.

The same test setup is also prepared for C57/BL6 mice as a control.

### 3. Gait Analysis

In the seven days preceding the gait test, Csf1r^{P853T/+} mice are trained. These mice are allowed to freely run from one side of a corridor to the other, with one session per day, consisting of 6 runs per session. As training progresses, the exploratory behavior of Csf1r^{P853T/+} mice within the corridor decreases. By the seventh day, most Csf1r^{P853T/+} mice can traverse the corridor continuously without pauses. Mice that still cannot cross the corridor without interruptions are excluded from the experiment. On the eighth day, Csf1r^{P853T/+} mice are placed in the corridor, and their gait is recorded and measured as they freely run from one side to the other for 4 runs.

The same test setup is also arranged for C57/BL6 mice as a control.

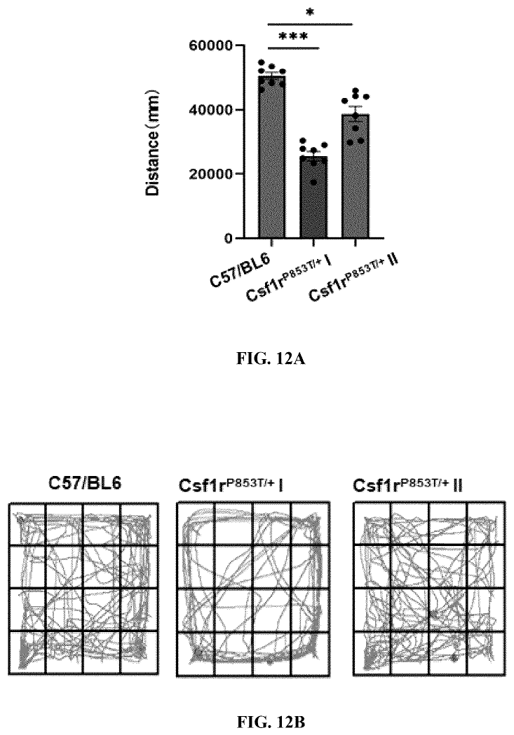

As shown in FIGS. 12A and 12B, the open-field test results indicate that both Csf1r^{P853T/+} type I and Csf1r^{P853T/+} type II mice exhibit a significant reduction in total traveled distance compared to wild-type mice. As shown in FIG. 14B, the rotarod experiment results reveal that Csf1r^{P853T/+} type I mice significantly decrease their time spent on the rod, while Csf1r^{P853T/+} type II mice show no significant reduction. As shown in FIG. 15B, the gait analysis results demonstrate that both Csf1r^{P853T/+} type I and Csf1r^{P853T/+} type II mice exhibit a significant increase in the distance between their hind paws when compared to wild-type mice.

### Example 4: Pathological Expression of Csf1r in Csf1r^{P853T/+} Mice

### 1. Immunohistochemistry Experiment

Following the completion of behavioral tests, the mice from each group are anesthetized. A 30 mL injection of normal saline is administered through the left ventricle of the heart, followed by systemic fixation with 4% PFA. The mice are then decapitated, and the entire brain is collected. The whole brain is sequentially immersed in 15%, 20%, and 30% sucrose solutions and subsequently frozen-sectioned to obtain 25 km-thick slices. The slices are subjected to a 15-minute incubation in 3% H₂O₂, followed by washing with PBS three times. Subsequently, after a 30-minute incubation with normal blocking serum, the primary antibody (Servicebio, GB11581) is added and incubated overnight at 4° C. with gentle agitation. The slices are then washed with PBS three times, followed by a 1-hour incubation at room temperature with the secondary antibody (Servicebio, GB23303). After another three washes with PBS, visualization of CSF1R expression in the striatum area of the mice from each group is achieved using the diaminobenzidine (DAB) method. The expression is observed under a light microscope.

### 2. Hematoxylin and Eosin (HE) Staining

After the completion of behavioral tests, the mice from each group are anesthetized. A 30 mL injection of normal saline is administered through the left ventricle of the heart, followed by systemic fixation with 4% PFA. The mice are then decapitated, and the entire brain is collected. The whole brain is sequentially immersed in 15%, 20%, and 30% sucrose solutions and subsequently frozen-sectioned to obtain 25 km-thick slices. These slices are fixed in 4% paraformaldehyde and allowed to air dry naturally. The subsequent steps for HE staining are as follows:

| | | |
|---|---|---|
| Distilled water | 1 | minute |
| Hematoxylin staining solution | 5-15 | minutes |
| Brief rinse under running water to | 1-3 | seconds |
| remove excess hematoxylin | | |
| 1% hydrochloric acid in ethanol | 1-3 | seconds |
| Brief rinse under running water | 10-30 | seconds |
| Eosin staining solution | 10-30 | seconds |
| Rinse under running water | 10-15 | minutes |
| Distilled water rinse | 1-2 | seconds |
| 0.5% alcoholic eosin solution | 1-3 | minutes |
| Distilled water rinse | 1-2 | seconds |
| 95% ethanol | 3-5 | minutes |
| Xylene (I) | 5 | minutes |
| Xylene (II) | 5 | minutes |

Mount with neutral mounting medium Results: Cytoplasm appears red, and cell nuclei appear blue-purple.

### 3. Luxol Fast Blue (LFB) Staining

After the completion of behavioral tests, the mice from each group are anesthetized. A 30 mL injection of normal saline is administered through the left ventricle of the heart, followed by systemic fixation with 4% PFA. The mice are then decapitated, and the entire brain is collected. The whole brain is sequentially immersed in 15%, 20%, and 30% sucrose solutions and subsequently frozen-sectioned to obtain 25 km-thick slices. These slices are immediately fixed in 4% paraformaldehyde and allowed to air dry naturally. Following rinsing with distilled water, the sections are immersed in a 0.1% LFB (Luxol Fast Blue) solution, sealed, and left to soak at 60° C. for 8-16 hours. Afterward, they are rinsed again with distilled water and then immersed in 95% alcohol. Subsequently, a 0.05% lithium carbonate solution is used for staining, with the sections being stained for at least 10 seconds. Further differentiation is achieved by continued exposure to 70% alcohol until a clear distinction between gray and white matter is observed under the microscope. Following a rinse with distilled water, a few drops of a 0.25% cresyl violet solution mixed with ice acetic acid staining solution are applied for a 10-minute secondary staining, and the color is further differentiated using 70% alcohol until the cell nuclei and Nissl bodies appear in red.

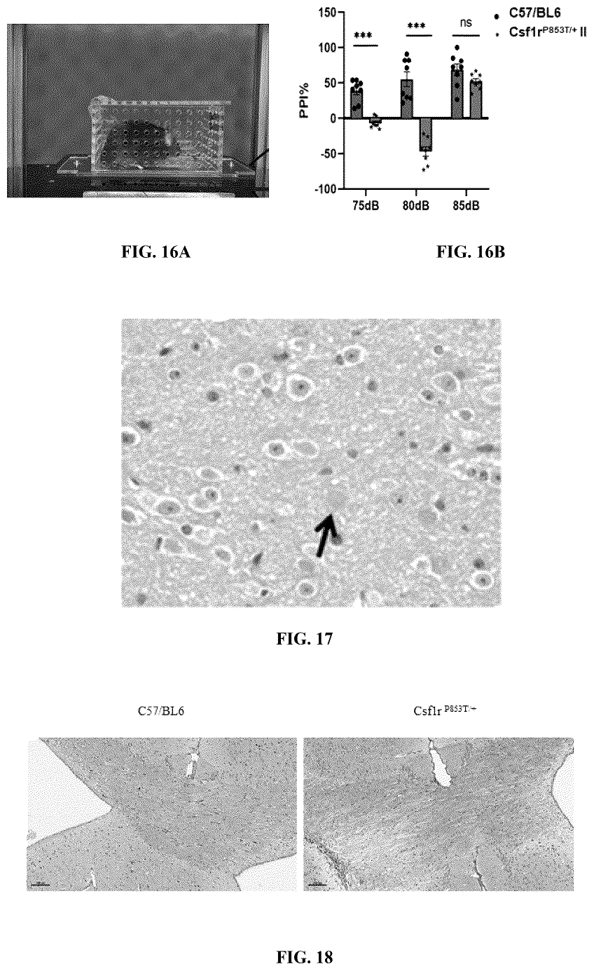

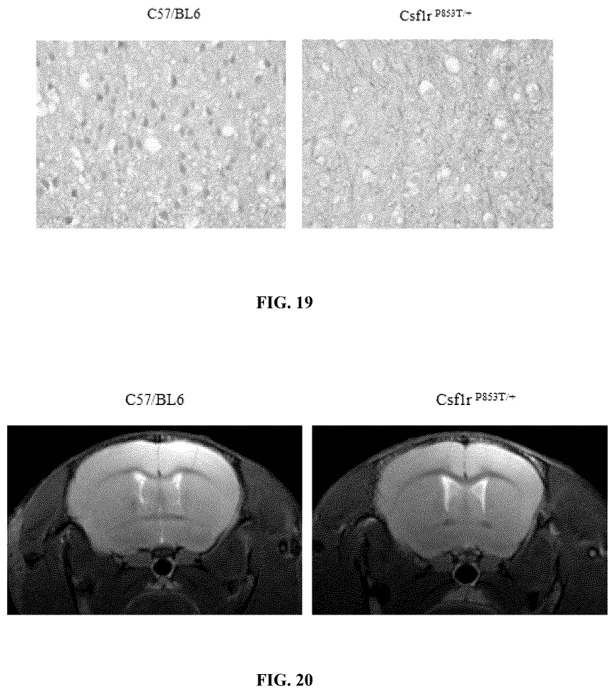

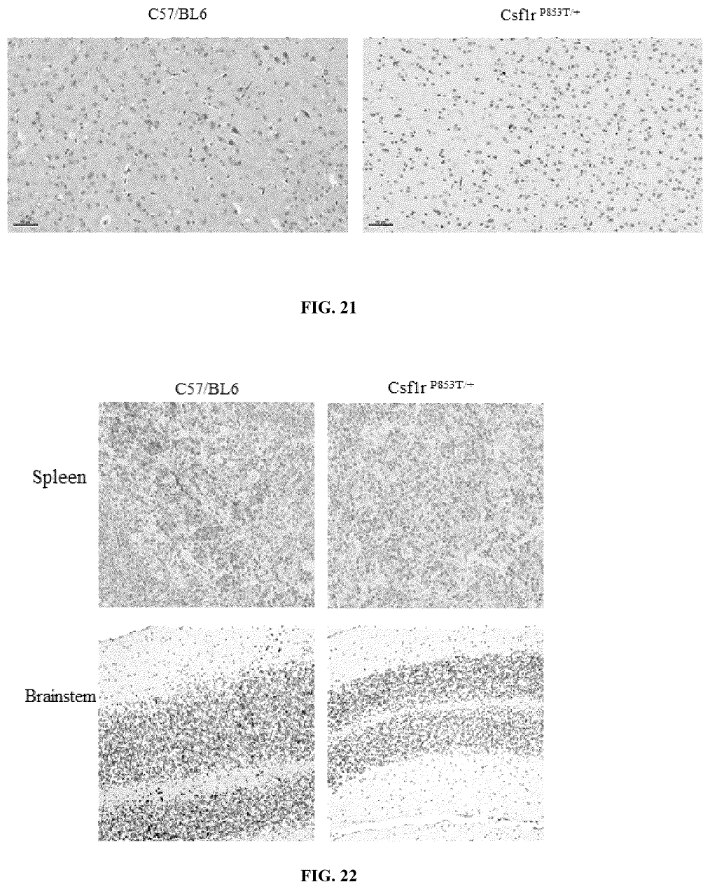

As shown in FIG. 22, compared to wild-type mice, Csf1r^{P853T/+} mice exhibit a significant reduction in Csf1r-positive cells in the spleen and brainstem. As shown in FIGS. 17, Csf1r^{P853T/+} mice display extensive white matter degeneration with typical axonal spheroid-like changes. As shown in FIGS. 18 and 20, compared to wild-type mice, Csf1r^{P853T/+} mice exhibit demyelination, thinning of the corpus callosum, enlargement of ventricles. Luxol Fast Blue (LFB) staining, as shown in FIG. 19, shows lipid and phospholipid-filled macrophages in Csf1r^{P853T/+} mice, which are absent in wild-type mice. FIG. 21 shows a significant increase in CD68-positive cells in Csf1r^{P853T/+} mice compared to wild-type mice.

What is claimed is:

1. A mutated Csf1r (Colony stimulating factor 1 receptor) gene, wherein the mutated Csf1r gene is obtained by changing 2557th nucleotide of a Csf1r gene from C to A; the mutated Csf1r gene is shown in SEQ ID NO: 1.

2. A mutated Csf1r protein, wherein the mutated Csf1r protein is encoded by the mutated Csf1r gene of claim 1; the mutated Csf1r protein is obtained by substituting proline with threonine at the 853rd amino acid position of a Csf1r protein; the amino acid sequence of the mutated Csf1r protein is shown in SEQ ID NO: 2.

3. An expression cassette, recombinant virus, recombinant cell, recombinant bacteria, or recombinant vector, comprising the mutated Csf1r gene of claim 1.

4. A method for constructing a murine model with mutations in Csf1r gene, comprising the following steps:
(1) constructing a targeting vector, Csf1r^{P853T/+}, containing the mutated Csf1r gene of claim 1;
(2) electroporating the targeting vector Csf1r^{P853T/+} obtained in step (1) into mouse embryonic stem cells and verifying positive clones;
(3) injecting the verified positive mouse embryonic stem cells from step (2) into blastocysts and transplanting the blastocysts into the uteri of female mice to obtain F0 generation mice;
(4) breeding the F0 generation mice obtained in step (3) with mice that express Cre enzyme in a tissue-specific manner and selecting F1 generation heterozygous mice that carry both the point mutation in step (1) and the Cre gene in their genomes;
(5) pairing two heterozygous F1 generation mice obtained in step (4) and selecting F2 generation homozygous mice, thereby obtaining the desired murine model.

5. The method according to claim 4, wherein the mice that express Cre enzyme specifically in tissues are mice that express Cre enzyme in macrophages.

6. The method according to claim 4, wherein the F2 generation homozygous mice are mice afflicted with brain diseases caused by microglial dysfunction.
